(19) United States
(12) Patent Application Publication
(10) Pub. No.: US 2003/0233095 A1
(43) Pub. Date: Dec. 18, 2003

(54) Device and method for attaching soft tissue to bone

(76) Inventors: Mark G. Urbanski, Sarasota, FL (US); Alberto Bauer, Marbella/Malaga (ES); Jose Lopez, Bradenton, FL (US)

Correspondence Address: RATNERPRESTIA, P O BOX 980, VALLEY FORGE, PA, 19482-0980, US

(21) Appl. No.: 10/167,586

(22) Filed: Jun. 12, 2002

(51) Int. Cl. A61B 17/08

(52) U.S. Cl. 606/72; 606/151; 606/232

(57) ABSTRACT

A device, system, and method for attaching soft tissue to bone is provided. The system includes an anchoring device and delivery device which allow a surgeon to achieve two different objectives during reattachment of tissue to bone. The system allows grasping and manipulation of the tissue to achieve proper location and tension on the tissue, and also attachment of the tissue to the bone after the desired location and tension are achieved. The anchoring device comprises a base, means for anchoring the device in bone, and at least two tissue grabbing members. Also included is a method of using the system to reattach soft tissue to bone.

### FIELD OF THE INVENTION

The present invention relates to medical devices and more specifically to a device and method for attaching soft tissue to bone.

### BACKGROUND OF THE INVENTION

There are several devices and methods known for attaching (or reattaching) soft tissue to bone. These devices and methods have been developed largely in response to the relatively common injuries associated with shoulders and knees whereby soft tissues, including ligaments, are torn or otherwise separated from the bone to which they are attached. Such an injury leads to chronic instability in the joint which often requires surgical intervention.

Surgical intervention conventionally involves the use of arthroscopic devices which use a cannula through which cameras and surgical devices are passed and used at the site of repair. These methods and devices have been designed for low trauma and faster recovery time for the patient.

Through the cannula, in addition to visualization devices such as cameras, various tools have been developed to reattach the torn soft tissue to the bone. Various anchors have been devised for attaching the torn tissue to the bone. One particular technique involves the insertion of an anchor into the bone. The anchor inserted either has sutures attached or means for attaching sutures to the anchor. The sutures are connected to the torn tissue and then tightened to allow contact of the tissue to the bone. The tissue and bone eventually reattach through natural healing process.

Such methods, however, have drawbacks. One such drawback is the fact that a surgeon must often use sutures to attach tissue to bone. Another such drawback is that the “pull-out strength” is often lower than desired. “Pull-out strength” is defined qualitatively as the force necessary to pull the anchor out of the bone to which it has been attached. Yet another drawback relates to “break-away strength.” As noted above, much of the prior art relies on sutures, which introduce another potential weakpoint. “Break-away strength” is defined qualitatively as the force necessary to break the suture. Still yet another drawback of the prior art is that the surgeon must use one device for locating and moving the torn soft tissue to the place of reattachment and a second tool or device for actually attaching the tissue. This is especially deleterious because the degree of stretching, or tautness, of the tissue at the time of reattachment must be precise to achieve proper healing and functionality of the joint after healing. Thus, the surgeon must be able to adjust the amount of tension placed on the ligament just prior to its reattachment. Having to use two different devices during placement, therefore, can lead to longer surgery and generally more room for error in tissue reattachment.

### SUMMARY OF THE INVENTION

The present invention includes devices, systems, and methods for attaching soft tissue to bone. The system allows the surgeon to achieve two different objectives during reattachment of the tissue to the bone. The same system allows grasping and manipulation of the tissue to achieve proper location of, and tension on, the tissue, and also attachment of the tissue to the bone after the desired location and tension are achieved. The system is comprised of an anchoring device and delivery device. The anchoring device, in its simplest embodiment, comprises a base and at least two members, each member having a base end and a tip end with a grasping region disposed between the tip and the base end, the base end connecting each member to the base.

A preferred embodiment of the anchoring device comprises a base, an overcenter toggle lock expandable from a collapsed position at which the device can be inserted into bone, to an overcenter stable expanded position to lock the anchoring device within the bone, and at least two tissue-grabbing members. The tips of the members are essentially like two opposing jaws of a pliers, which together grasp the tissue and allow the surgeon to push the members, along with the grasped tissue, down into a hole in the bone. The locking mechanism is then activated to anchor the device and tissue within the bone. The tissue-grabbing members (and their associated base and locking mechanism) are then released from the delivery device and left in place. The tissue and bone are allowed to grow together during the healing process. In a preferred embodiment, the device is biodegradable.

Also included as a part of the present invention is a system including the anchoring device and a delivery device for attaching soft tissue to bone comprising means for expanding the anchoring device members from an unexpanded position to an expanded position within the bone.

A preferred system comprises an applicator having a distal and proximal end and a push rod slidably and removably disposed within the applicator. The distal end of the applicator is moveable between a first position for holding the anchoring device and a second position for releasing the anchoring device. The push rod is slidable between a retracted position which corresponds to the first position of the applicator, and a forward position which corresponds to the second position of the applicator. Also included in a preferred embodiment of this system is an outer sleeve slidably and removably disposed around the applicator.

The method of the present invention comprises the steps of grasping a portion of soft tissue with the distal end of a device, inserting the device along with the grasped portion of soft tissue into a hole in a bone, and anchoring the device within the hole into which it was inserted by expanding the device. The delivery device is then removed and the tissue is allowed to grow and reattach to the bone.

### BRIEF DESCRIPTION OF THE DRAWING

The features of the invention believed to be novel and the elements characteristic of the invention are set forth with particularity in the appended claims. The figures are for illustration purposes only and are not drawn to scale. The invention itself, however, both as to organization and method of operation, may best be understood by reference to the detailed description which follows taken in conjunction with the accompanying drawings in which:

FIG. 1A is a side view of a cross section of an anchoring device in its unexpanded position according to the present invention;

FIG. 1B is a side view of a cross section of the anchoring device of FIG. 1A in its expanded, locked position according to the present invention;

FIG. 2A is a side view of a cross section of an anchoring device disposed on the distal end of a delivery device in accordance with the present invention;

FIG. 2B is the view shown in FIG. 2A except with the sleeve moved forward to close the jaws of the anchoring device;

FIG. 2C is a side view of a cross section of an anchoring device being expanded by a push rod in accordance with the present invention;

FIG. 2D shows the removal of a delivery device from the anchoring device;

FIG. 2E shows the distal end of an applicator in accordance with the present invention;

FIG. 3A is an angled view of an anchoring device according to the present invention;

FIG. 3B is an angled view of an alternative anchoring device according to the present invention;

FIG. 3C is an angled view of an alternative embodiment to the device shown in FIG. 3B, but where no bone engaging barbs are present on the members;

FIG. 3D is an angled view of another alternative anchoring device according to the present invention;

FIG. 3E shows an alternative embodiment of part of the system of the present invention in which a conical shaped screw expander 330 is disposed within the anchoring device;

FIG. 3F shows conical shaped screw expander 330 rotated distally to expand an anchoring device;

FIG. 3G shows yet another embodiment, wherein a smooth, conical shaped expander rod is used to expand the anchoring device;

FIG. 3H shows still yet another embodiment, wherein the expander means and applicator are screwed together;

FIG. 4 is a cross section of soft tissue attached to a bone;

FIG. 5 is a cross section of soft tissue torn from a bone to which it was attached;

FIG. 6 shows the drilling of the bone for which reattachment of torn soft tissue is desired in accordance with the present invention;

FIG. 7 shows the removal of a drill after drilling of the bone for which reattachment of torn soft tissue is desired in accordance with the present invention;

FIG. 8 shows the first step of the grasping of soft tissue in accordance with the present invention;

FIG. 9 shows the sleeve causing closure of the jaws of the anchoring device to grasp soft tissue in accordance with the present invention;

FIG. 10 shows movement of the soft tissue in accordance with the present invention;

FIG. 11 shows initial placement of soft tissue in the hole in the bone in accordance with the present invention;

FIG. 12 shows insertion of soft tissue into the hole in the bone in accordance with the present invention;

FIG. 13 shows a close up view of that shown in FIG. 12 but with the removal of the sleeve in accordance with the present invention;

FIG. 14 shows the push rod moving distally to expand the anchoring device of the present invention;

FIG. 15 shows the push rod completely forward, the anchoring device fully expanded, and the release of the anchoring device from the distal end of the applicator;

FIG. 16 illustrates the removal of the delivery device after expansion of the anchoring device;

FIG. 17 shows the final placement of anchoring device within the bone with the soft tissue anchored therein;

FIG. 18 shows one embodiment of the method of the present invention to tighten soft tissue tension prior to its insertion into bone by rotating the delivery device while the soft tissue is grasped within the anchoring device,

FIG. 19 shows an alternative embodiment of the present invention where two anchoring devices are used, one placed sequentially after the first; and

FIG. 20 shows still another alternative embodiment of the present invention where a plug is used to insert additional tissue into the first placed anchoring device in accordance with the present invention.

### DETAILED DESCRIPTION OF THE INVENTION

The present invention includes devices, systems, and methods for reattaching soft tissue to bone. Although many places in a human or animal body have tissue to bone connection, the present invention is particularly well suited for repairs to the shoulder or knee joints such as reconstructing the anterior cruciate ligament or repairing a dislocated shoulder or torn rotator cuff.

Generally, the present invention includes an anchoring device which allows grasping and manipulation of the tissue to achieve proper location and tension on the tissue, and also attachment of the tissue to the bone after the desired location and tension are achieved. The anchoring device, in its simplest embodiment, comprises a base and at least two tissue-grabbing members, each member having a base end and a tip end with a grasping region disposed between the tip, and the base end, the base end connecting each member to the base.

A preferred embodiment of the anchoring device comprises a base, an overcenter toggle lock expandable from a collapsed position at which the device can be inserted into bone, to an overcenter stable expanded position to lock the anchoring device within the bone, and at least two tissue-grabbing members connected to the base. The tissue-grabbing members are essentially like opposing jaws of a pliers, which together grasp the tissue and allow the surgeon to push the members (jaws) along with the grasped tissue, down into a hole in the bone. The locking mechanism is then activated to anchor the device and tissue within the bone. The members (and their associated base and locking mechanism) are then released from a delivery device and left in place. The tissue and bone are allowed to grow together during the healing process. In a preferred embodiment, the device is biodegradable.

In one embodiment, the tissue-grabbing members also connect the base to the expandable toggle lock to transmit forces to the toggle lock to maintain it in the stable overcenter position. In another embodiment, the overcenter toggle lock and the tissue-grabbing members are separately attached to the base, and are disposed perpendicular to each other.

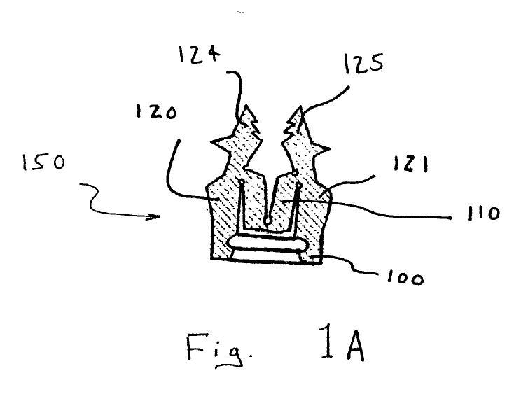

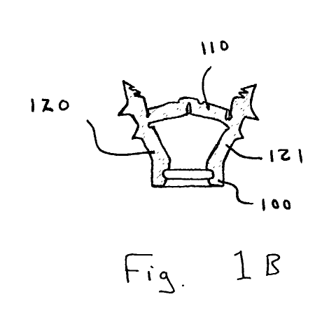

FIG. 1A shows a cross section of a device in accordance with the present invention. Base 100 is connected to overcenter toggle lock 110 by support members 120 and 121. Overcenter toggle lock 110 is expandable from a collapsed position at which the device can be inserted into bone, to an overcenter stable expanded position to lock the fastener within the bone. Tissue-grabbing members 124 and 125 are shown with teeth. Together these elements define anchoring device 150.

FIG. 1B shows the same device in its expanded, locked position. The device may have more than two members, but the preferred embodiment, as shown in FIGS. 1A and 1B, has only two members. The members connecting the base and the expandable toggle lock transmit internally directed axial forces to the toggle lock to maintain it in the stable overcenter position once expanded.

The device may be made from a number of different materials, so long as the material is pliable enough to allow movement between the unexpanded and expanded positions. Preferably, the anchoring device is made from a biodegradable polymer such as a polylactide based copolymer. Preferred among these are poly(l-lactide) (PLLA) and poly(dl-lactide) (PDLLA). More preferred are blends of these polymers, including a 70%PDLLA/30%PLLA blend. Other suitable, biodegradable polymers, exhibiting sufficient elasticity and strength, may be used.

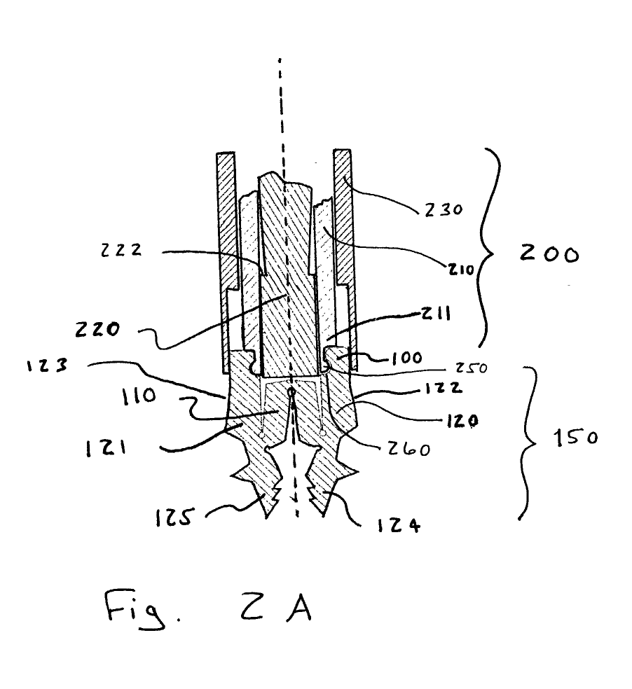

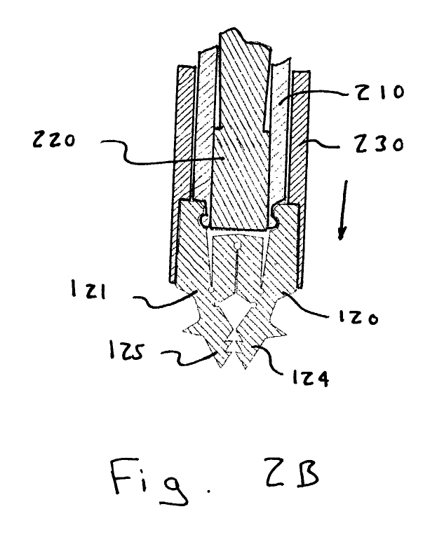

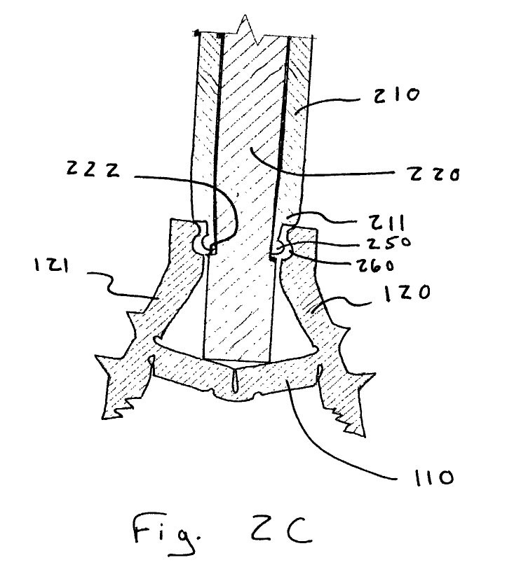

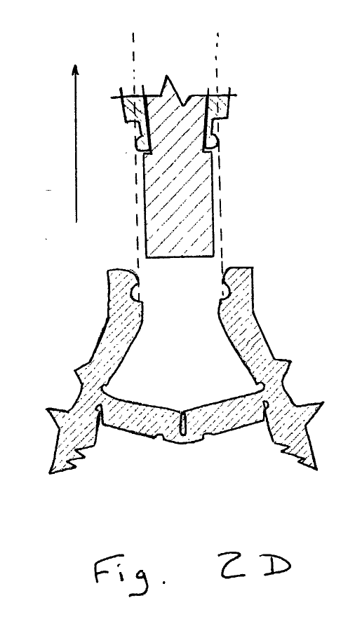

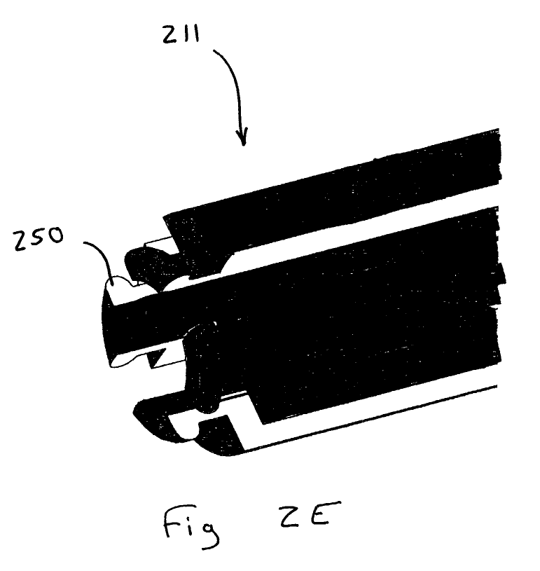

FIG. 2A shows anchoring device 150 removably attached to the end of delivery device 200. This embodiment of delivery device 200 is comprised of applicator 210 having a distal end 211, a push rod 220 slidably and removably disposed coaxially within the applicator, and sleeve 230 disposed around applicator 210. Each element (applicator 210, push rod 220, and sleeve 230) is longitudinally slideable with respect to the other elements along a common, central axis, indicated by the dotted line in FIG. 2A.

Distal end 211 of applicator 210 is constructed to be biased inward toward the central axis such that push rod distal end 221 applies an outward force with respect to applicator distal end 210 so long as push rod distal end 221 is disposed as shown in FIG. 2A. FIG. 2E shows a view of distal end 211 of applicator 210. Push rod 220 has a groove 222 formed around its circumference as shown in FIG. 2A. Groove 222 is disposed around the circumference of push rod 220 so that when push rod 220 is pushed beyond the point where push rod 220 keeps applicator distal end 211 outwardly disposed, applicator distal end 211, under force of its preconstructed inward bias, moves inward. This will allow the release of device 150, as shown in FIGS. 2C and 2D, and as discussed in more detail below.

Thus, the interaction between push rod 220 and applicator 210 serves to control the device both by holding it in place before release, and allowing its release when desired by the surgeon controlling delivery device 200. Moreover, the distal end of the applicator is moveable between a first position for holding the anchoring device and a second position for releasing the anchoring device and the push rod is slideable between a retracted position which corresponds to the first position of the applicator, and a forward position which corresponds to the second position of the applicator.

Sleeve 230 serves a different purpose. Sleeve 230 provides a means for using device 150 as a pliers-like tool for grasping and moving soft tissue. Sleeve 230 is slideable between a retracted position, as shown in FIG. 2A, where the support members 120 and 121 (and tissue-grabbing members 124 and 125) are allowed to be open, and a forward position, as shown in FIG. 2B, where support members 120 and 121 and tissue-grabbing members 124, 125, are pushed together into a closed, grasping position. This action is achieved by sliding sleeve 230 from its retracted position to its forward position, whereby a compressing force is exerted on support members 120 and 121 because of the outwardly sloped surfaces 122 and 123 along the outside surface of support members 120 and 121, as shown in FIG. 2A. This outward sloping is seen when device 150 is in its relaxed position. Specifically, each of the two members disposed opposite from each other across the central axis of the anchoring device has a compression region disposed at its proximal portion; in the case of FIG. 2A, between the base and the point where the overcenter toggle lock contacts the member. The compression region has a proximate end and a distal end, and an outer dimension spaced from the central axis. Moreover, the outer dimension of the compression region increases in magnitude from the proximal end of the compression region to the distal end of the compression region, creating an outwardly sloped surface as one moves from the base toward the distal end of the device.

Thus, as sleeve 230 is pushed to its forward position, it applies an inward force on support members 120 and 121, causing the closing of tissue-grabbing members 124 and 125, as shown in FIG. 2B, which close like the nose of a needle-nosed pliers.

After the device is used to grasp, move, and insert soft tissue into a hole in a bone using the sleeve and pliers functionality, the tissue can be anchored into the hole in the bone to which it was once attached. This is accomplished by removing the sleeve, and advancing the push rod forward. FIG. 2C shows sleeve 230 removed, with push rod 220 already advanced into its forward position. Because push rod 220 has been advanced to its forward most position, two resultant actions have occurred. First, overcenter toggle lock 110 has been forced open, against the resistive force of members 120 and 121. Because overcenter toggle lock 110 has been moved beyond a line perpendicular to the longitudinal axis of the device, and because it is still under the compressive, resistive force of support members 120 and 121, overcenter toggle lock 110 will serve to keep the device in this opened position. Second, because groove 222 of push rod 220 has moved below the distal end 211 of applicator 210, the inward bias of applicator 210 at its distal end 211 has caused distal end 211 to move inward, reducing its diameter. The reduction in diameter results in the ability for the delivery device to be removed from anchoring device 125. More specifically, and as shown in FIG. 2C, male protrusion 250 at the distal end 211 of applicator 210 moves inward, and out of, female groove 260 formed in base 100 of device 150. As shown in FIG. 2A, male protrusion 250 is pushed outward and into female groove 260 of base 100 when push rod 220 is in its withdrawn position. Moreover, when push rod 220 is pushed to a point where push rod groove 222 moves beyond the end of applicator 210, the applicator end moves inward, and releases the device. At that point, delivery device 200 can be withdrawn as shown in FIG. 2D, leaving the expanded anchoring device 150 in place within a hole in a bone.

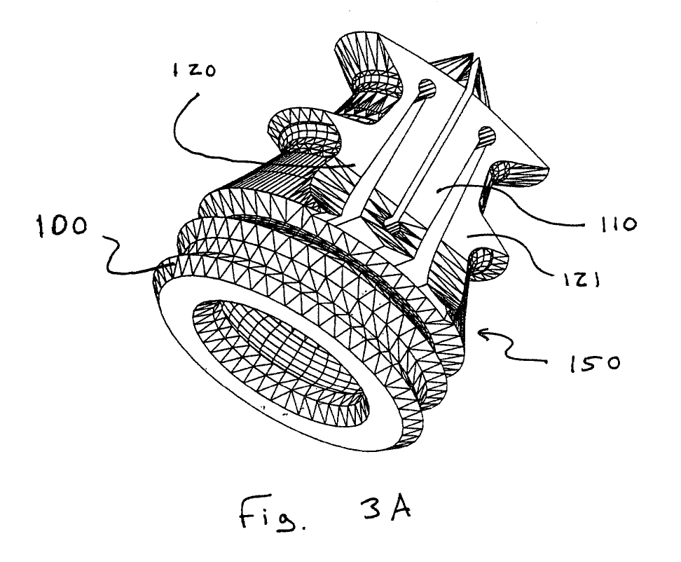

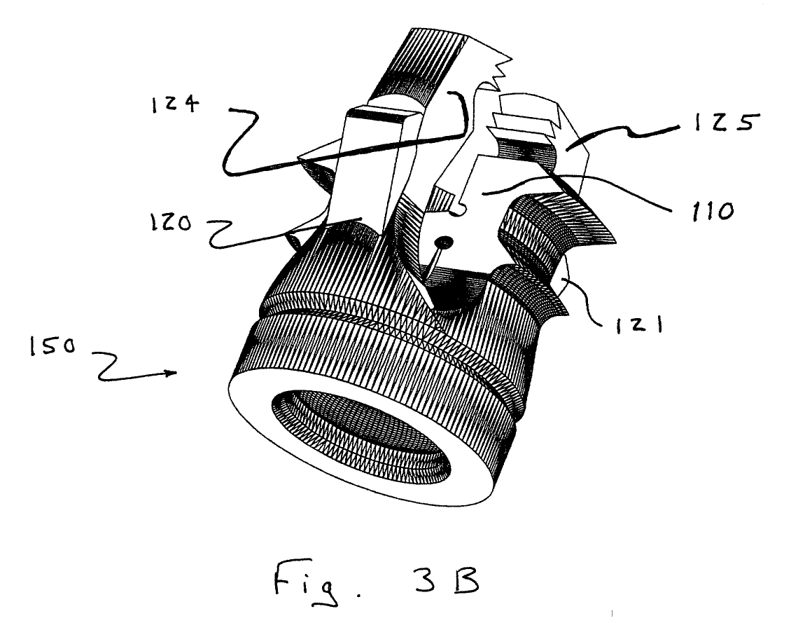

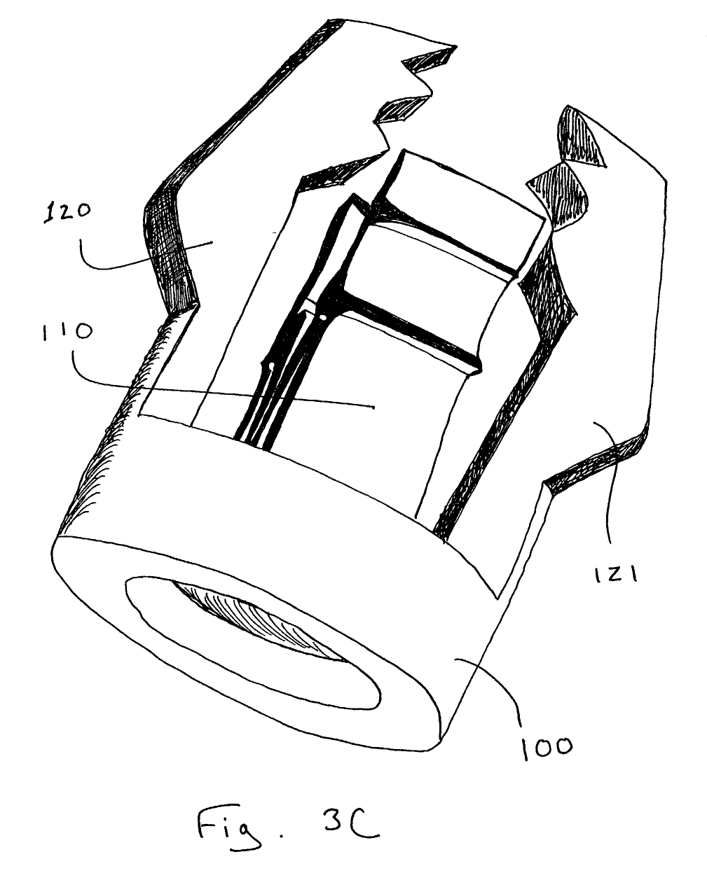

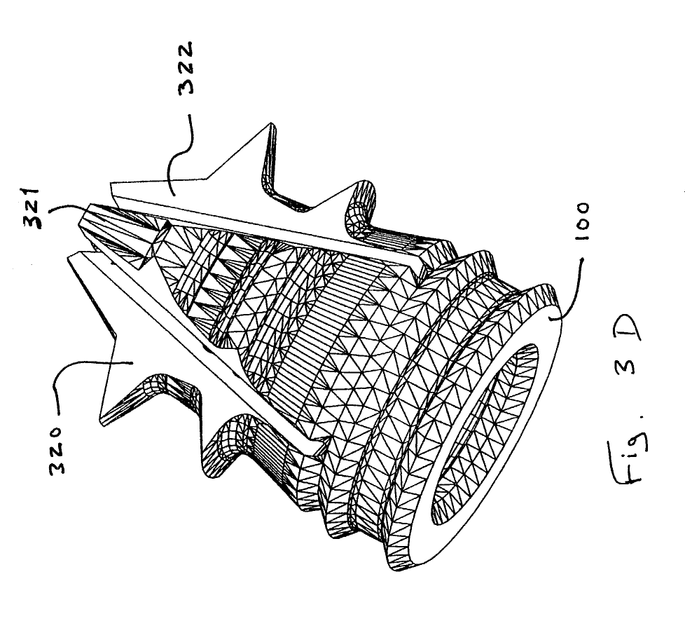

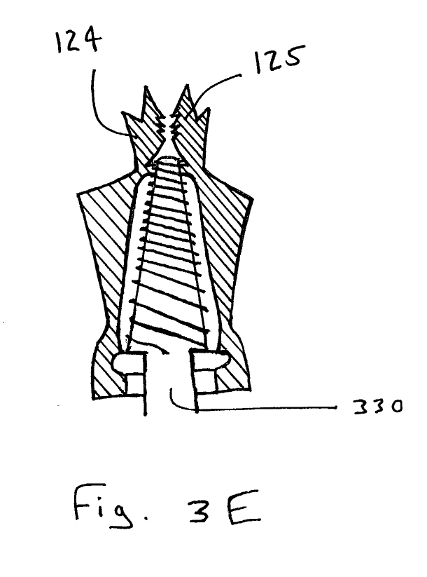

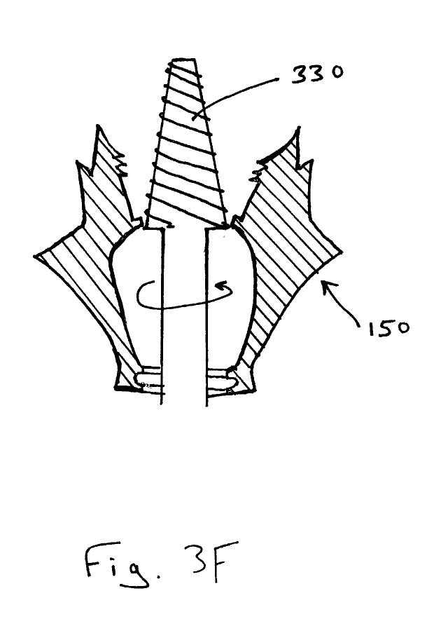

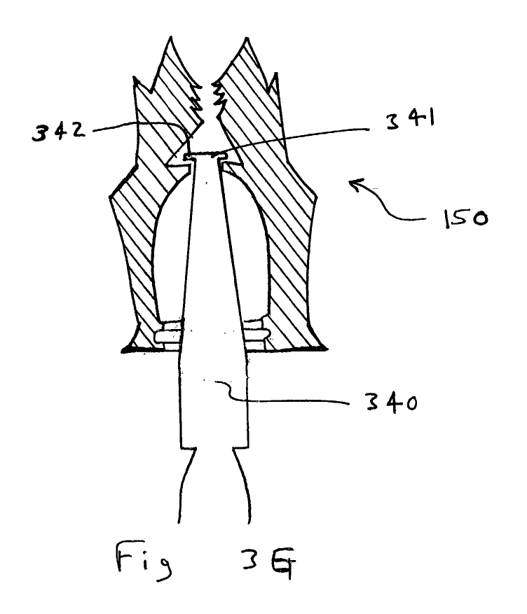

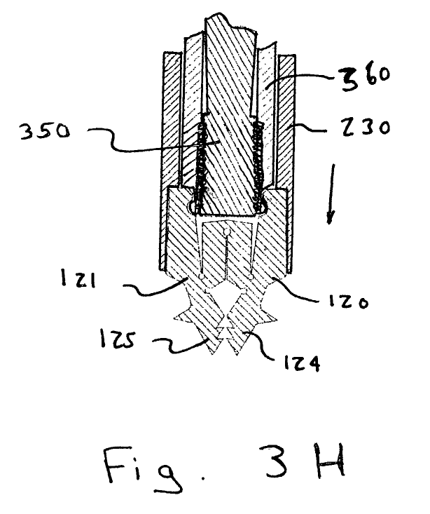

FIG. 3A shows one embodiment of anchoring device 150 according to the present invention. This view is similar to that which is shown in FIG. 1 in cross section, with overcenter toggle lock 110 disposed in the same plane as support members 120 and 121 and tissue-grabbing members 124 and 125.

FIG. 3B shows an embodiment of anchoring device 150 which is similar to that shown in FIG. 3A, but with overcenter toggle lock 110 disposed separately and perpendicular to support members 120 and 121. In this embodiment, the support members supporting the tissue-grabbing members 124, 124 are not used to impart forces on the overcenter toggle lock as shown in, for example, FIG. 3A. Instead, overcenter toggle lock is separate from support members 120 and 121 and tissue-grabbing members 124, 125.

FIG. 3C shows a preferred embodiment of the present invention which is similar to that shown in FIG. 3B, but which does not have bone engaging barbs on members 120 and 121. This is because, in this embodiment, the support members 120 and 121 are not expanded into bone.

FIG. 3D shows an embodiment of the present invention where four members 320, 321, 322, and 323 (323 not shown) are used. In this embodiment, the overcenter toggle lock could be connected to any two or more members.

FIG. 3E shows an alternative embodiment of part of the system of the present invention in which a conical shaped screw expander 330 is disposed within the anchoring device. In this embodiment, where screw expander 330 is screwably attached to the base of tissue-grabbing members 124, 125, a sleeve may not be necessary to actuate the gripping of tissue, because conical shaped screw expander 330 could be rotated proximally to draw jaws 124, 125 together to grasp tissue, and could then be rotated distally as shown in FIG. 3F to expand anchoring device 150. Alternative embodiments would include, however, use of an anchoring device which is constructed to have an inward bias whereby the jaws are set together, only to be opened under force of the screw expander. In such an embodiment, use of a sleeve would aide in holding tissue within the jaws. In either event, the distal end of conical shaped screw expander 330 could be detached from the delivery device and left in the bone along with anchoring device 150. Conical shaped screwed expander 330, in such a case, would also be comprised of a biodegradable material.

Yet another embodiment of the invention is shown in FIG. 3G which uses a smooth, conical shaped expander rod 340. This embodiment is similar to that disclosed in FIG. 3E, but does not use the rotated screw aspect. In this embodiment, distal end 341 of smooth conical shaped expander rod 340 would have an enlarged tip 342 which would be used to pull tissue-grabbing members 124, 125 together by pulling on smooth, conical shaped expander rod 340 proximally. Then, by moving smooth, conical shaped expander rod 340 distally, the conical shape would force tissue-grabbing members 124, 125 apart and into bone as described more fully throughout. The use of a sleeve in this embodiment would not be necessary.

These expansion means can be modified or combined in a number of different ways to achieve the same ultimate objective: expansion of the anchoring device. For example, the system illustrated in FIG. 3H shows the same system as that of FIG. 2B, except that push rod 220 and applicator 210 have been modified so that they are screwed together. In this embodiment, screw rod 350 is rotated and advanced through applicator 360. Other such expansion means can be envisioned within the scope of the present invention.

By combining the functionality of the expander means, applicator, and sleeve as described above in conjunction with the different anchoring devices described herein, the delivery device can be used to grasp, move, insert, and anchor soft tissue into a hole in a bone. One example of this method is addressed below.

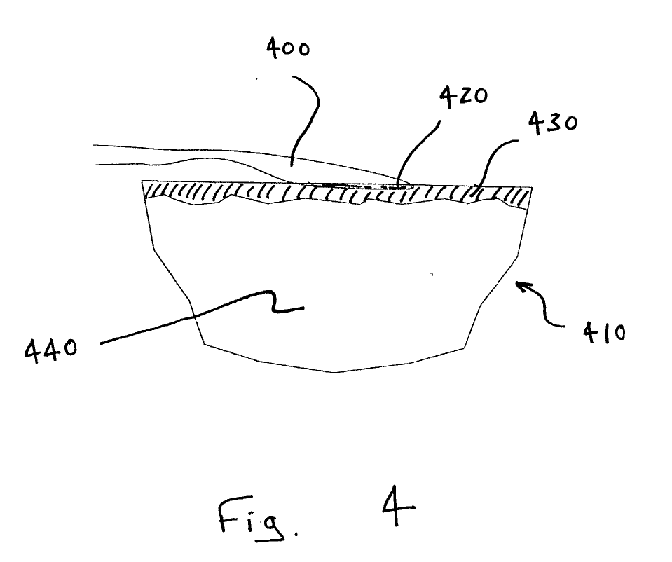

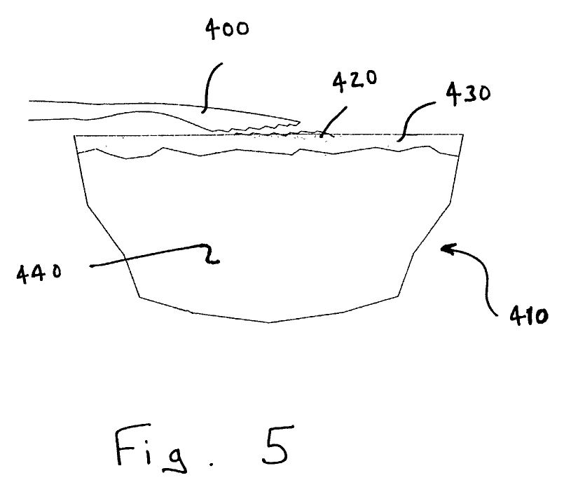

FIGS. 4 to 17 show a method according to the present invention. FIG. 4 shows a piece of normal soft tissue 400 attached to bone 410. Tissue ingrowth area 420 is shown where soft tissue 400 contacts a layer of cortical bone 430. Cancellous bone 440 (softer than the cortical bone) is shown in part below cortical bone 430. FIG. 5 shows soft tissue 400 torn from cortical bone 430.

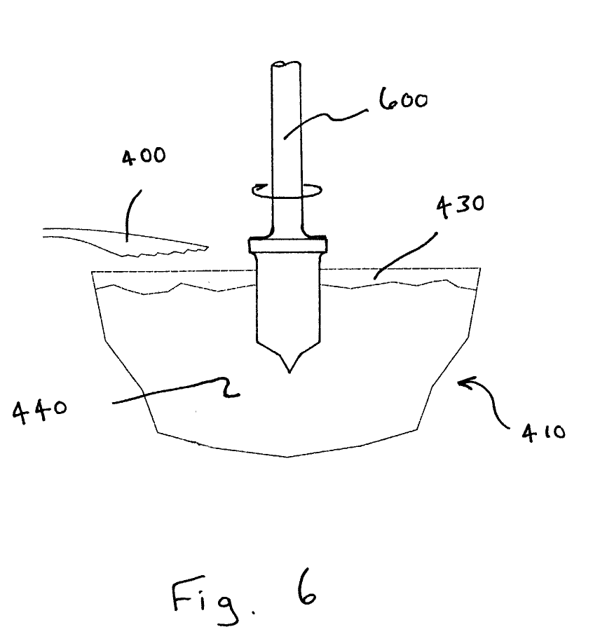

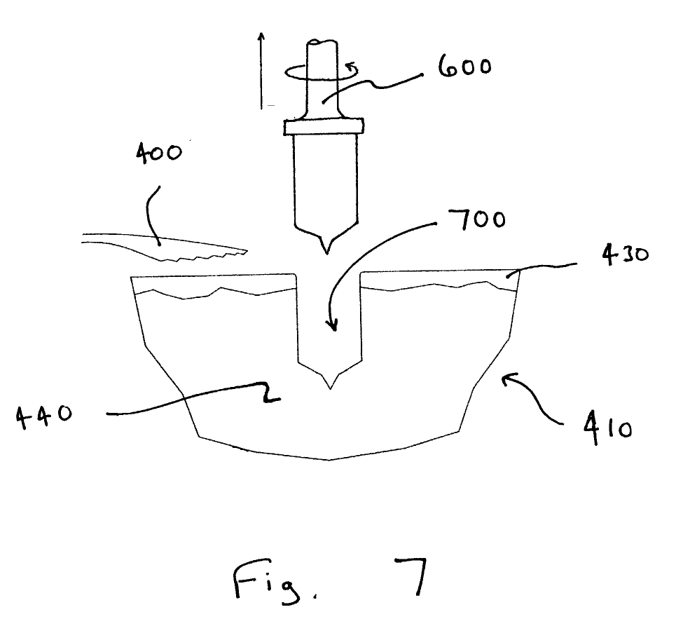

The first step in repairing the tissue after access to the site is achieved by the surgeon is to clean and prepare the bone surface area for drilling. FIG. 6 illustrates drill 600 penetrating cortical bone 430 and cancellous bone 440 to form hole 700, shown in FIG. 7. The types of drill bits and methods for accessing the affected area with drill 600 are well known by those skilled in the art. Important in this step is to insure that hole 700 is drilled to the proper depth. As will be seen more clearly below, anchoring device 150 must penetrate bone 410 to a depth sufficient to allow effective expansion and anchoring of device 150 along with soft tissue 400 which is forced into hole 700.

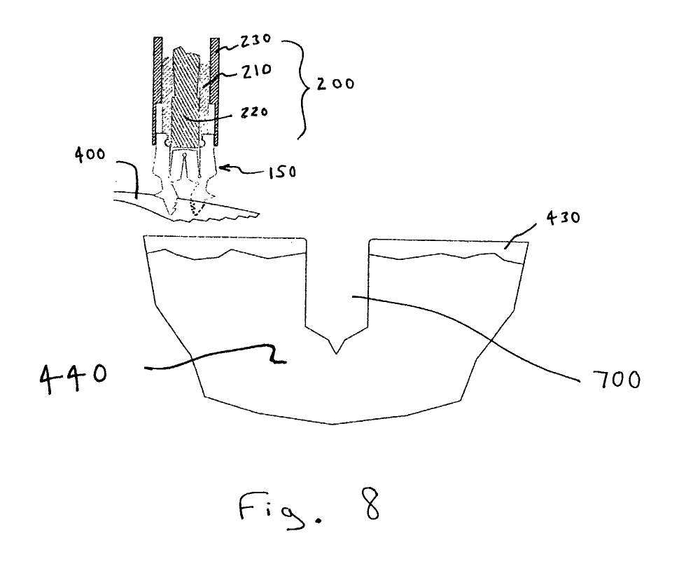

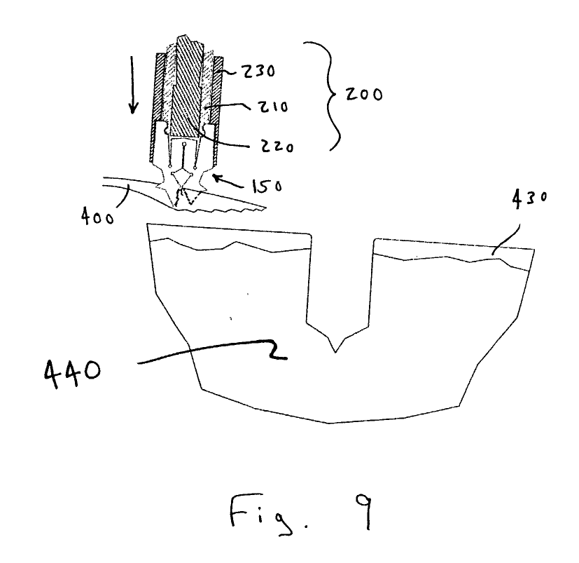

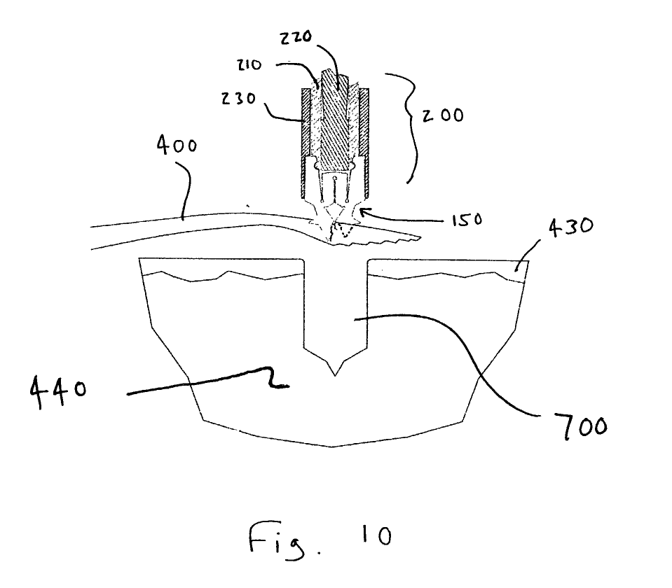

FIG. 8 shows the next step, namely inserting anchoring device 150 which is disposed on the distal end of delivery device 200. The surgeon locates soft tissue 400 for which repair is desired, then grasps soft tissue 400, as shown in FIG. 9, by sliding sleeve 230 distally to close tissue-grabbing members 124 and 125 around soft tissue 400. The grasped soft tissue 400, anchoring device 150, and delivery device 200 are then manipulated by the surgeon to position soft tissue 400 above hole 700 as shown in FIG. 10. When soft tissue 400 is pulled over hole 700, the soft tissue undergoes a force which tightens it, and may even stretch it. The surgeon can control the degree of taughtness in a variety of ways. Some of these ways are discussed in more detail below.

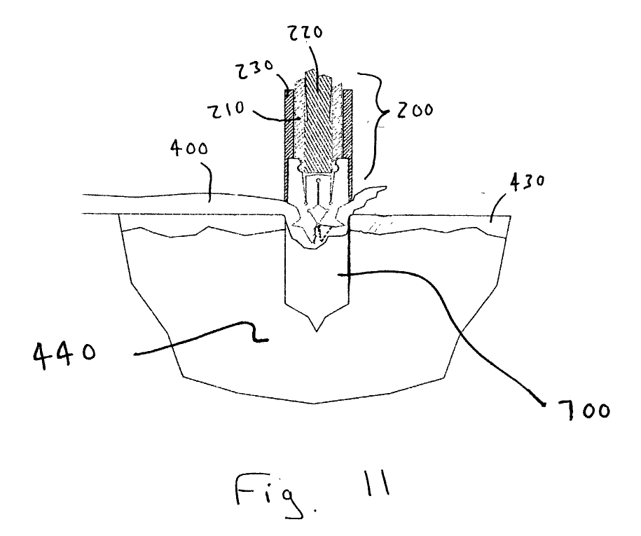

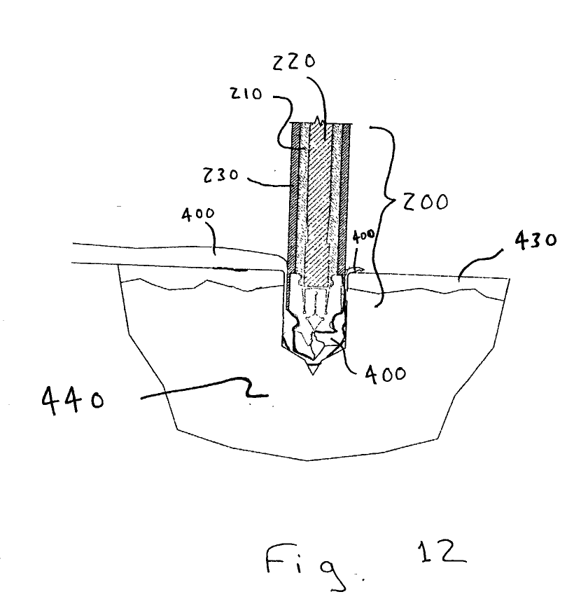

Once the surgeon decides to anchor a piece of soft tissue 400, the surgeon can push the system, including delivery device 200 and anchoring device 150, down into hole 700. FIG. 11 shows delivery device 200 and anchoring device 150 lined up above hole 700 with soft tissue 400 in place as the system begins its way into hole 700. FIG. 12 shows delivery device 200 and anchoring device 150 disposed down in hole 700. It is important that hole 700 is deep enough so that anchoring device 125 is sufficiently inserted into cancellous bone 440. Otherwise, proper expansion of anchoring device 150 in subsequent steps may not be achieved.

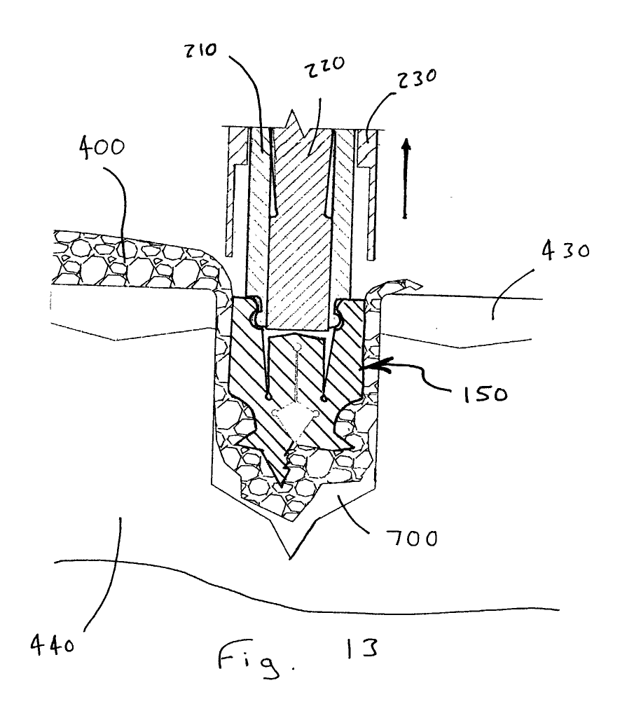

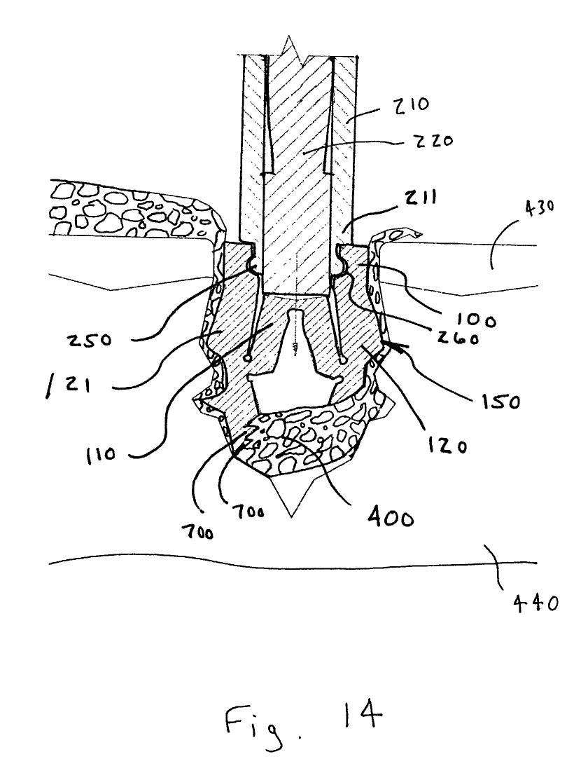

FIG. 13 is an expanded view of FIG. 12, but with the retraction of sleeve 230 illustrated. As sleeve 230 is retracted, anchoring device 150 does not open because it is compressed within hole 700 by cancellous bone 440 and soft tissue 400. FIG. 14 shows the effect of the surgeon advancing push rod 220 forward to begin opening anchoring device 150. It can be seen that male protrusion 250 at the distal end 211 of applicator 210 is still in its outward position, fitted within female groove 260 formed in base 100 of device 150. Thus, anchoring device 150 is still held tightly to the end of applicator 210. Because of the softness of cancellous bone 440, anchoring device 125 is able to open against the cancellous bone 440.

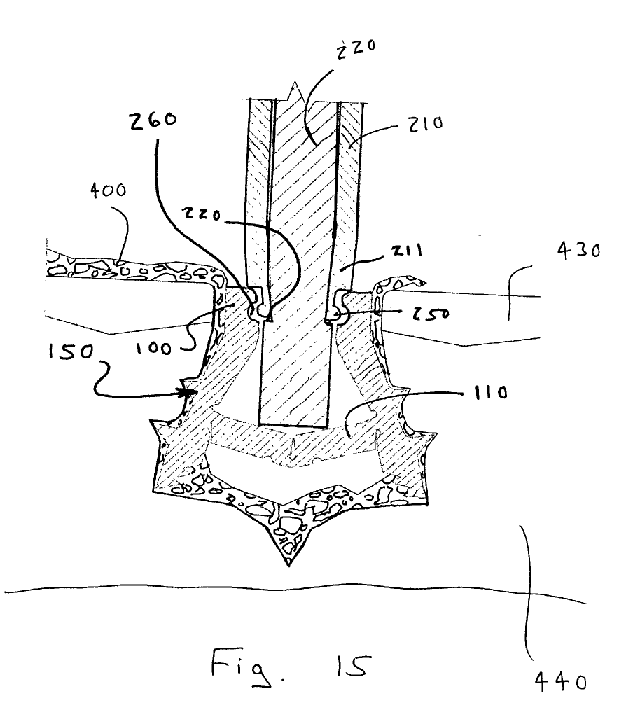

As push rod 220 is advanced by the surgeon, overcenter toggle lock 110 begins to spread, pushing support members 120 and 121 outwardly into cancellous bone 440. With the spreading of members 120 and 121, of course, comes the spreading of tissue-grabbing members 124 and 125. Push rod 220 is advanced until overcenter toggle lock 110 is pushed beyond a line parallel with the longitudinal axis of the system, as shown in FIG. 15. In the embodiment using the device as shown in FIG. 3C, for example, the expansion of overcenter toggle lock would not, of course, affect the tissue grabbing members 124, 125, allowing them to retain their grasp on soft tissue 400.

FIG. 15 also illustrates what happens between applicator 210 and device 150 when push rod 220 fully expands overcenter toggle lock 110. Because of the inward bias of distal end 211 of applicator 210 as described above, when push rod 220 is sufficiently advanced such that groove 222 of push rod 220 has moved below the distal end 211 of applicator 210, the inward bias of applicator 210 at its distal end 211 has caused distal end 211 to move inward, reducing its diameter. At this point, male protrusion 250 at distal end 211 of applicator 210 moves inward, and out of, female groove 260 formed in base 100 of device 150.

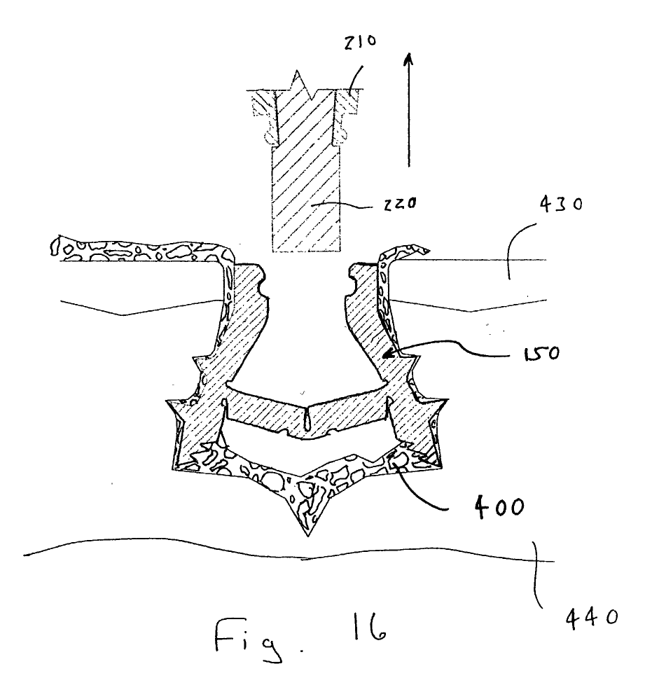

FIG. 16 shows the removal of push rod 220 and applicator 210 from device 150, which is now anchored, along with soft tissue 400, within cancellous bone 440 and cortical bone 430.

After a period of time for healing has passed, soft tissue 400 will have rejoined coritcal bone 430, and anchoring device 150 will biodegrade. Thus, there is no need for the surgeon to re-enter the area or otherwise return for additional adjustment or work in the future. All that will remain after healing is soft tissue attached to bone, with no metal or other foreign objects in place.

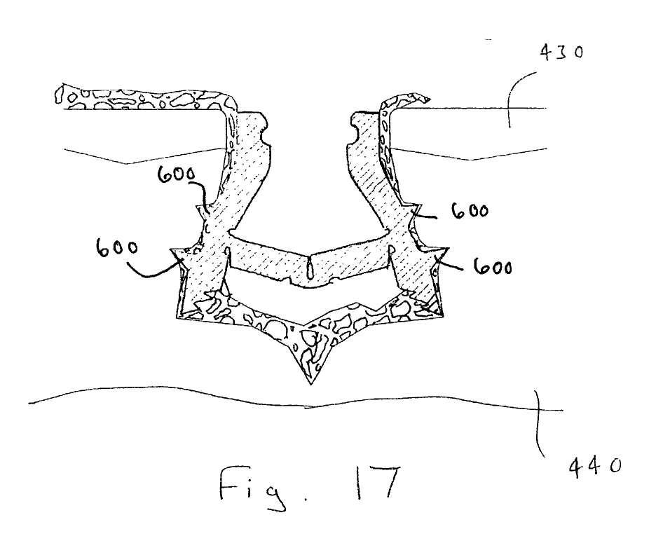

Aiding in the anchoring of device 150 are barbs 600 shown in various figures, including, for example, FIG. 17, which illustrates device 150 in place within a segment of bone. These barbs may be disposed on the outside of support members 120 and 121 to increase pull-out strength. In those embodiments, where support members 120, 121 are not expanded, such as when the device of FIG. 3C is used, barbs may suitably be placed on the outside of the supports of the overcenter toggle lock. Also included in a preferred embodiment are teeth 700 as shown in FIG. 14, for example, for aiding in grasping soft tissue 400.

As noted above, it is preferred that the surgeon be able to adjust the “taughtness” of the soft tissue, particularly in the case of ligament reattachment, prior to anchoring the tissue into the bone. This can be achieved in a number of ways, some of which are discussed below.

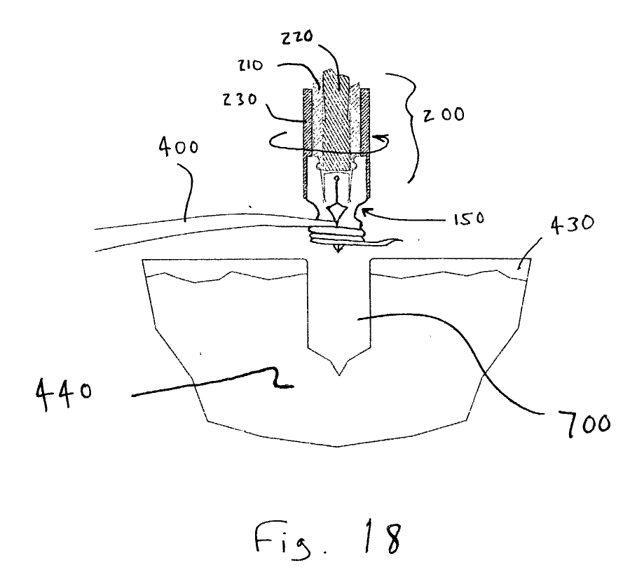

The surgeon can, after initially grasping a piece of soft tissue, twist the entire device, or rotate it, around its central axis, in order to tighten the tissue prior to inserting it into the prepared hole in the bone. This is illustrated in FIG. 18.

Alternatively, the surgeon can grasp, move, partially insert soft tissue into the hole, and then release the tissue and move the device back to regrasp additional tissue and reinsert that tissue over top of the originally inserted tissue. This can be continued until the desired tension in the soft tissue remaining outside of the hole is achieved.

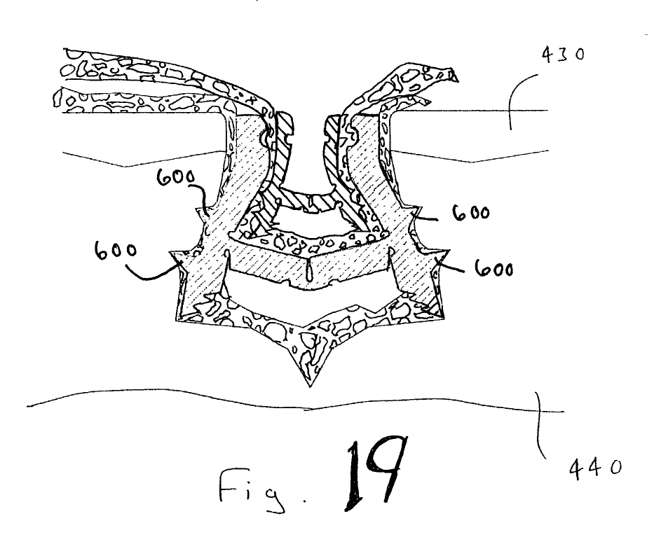

Another way to achieve the desired tension is to anchor a piece of soft tissue into a hole as described above, remove the delivery device and return with a second anchoring device to repeat the process while tightening the tissue the second and subsequent times. In such a case, each time the tissue is inserted, it could be inserted into a different hole. Alternatively, because the base of anchoring device 150 is open in its center, progressively smaller anchoring devices could be used and each inserted into the last-placed anchoring device. Such a system is illustrated in FIG. 19.

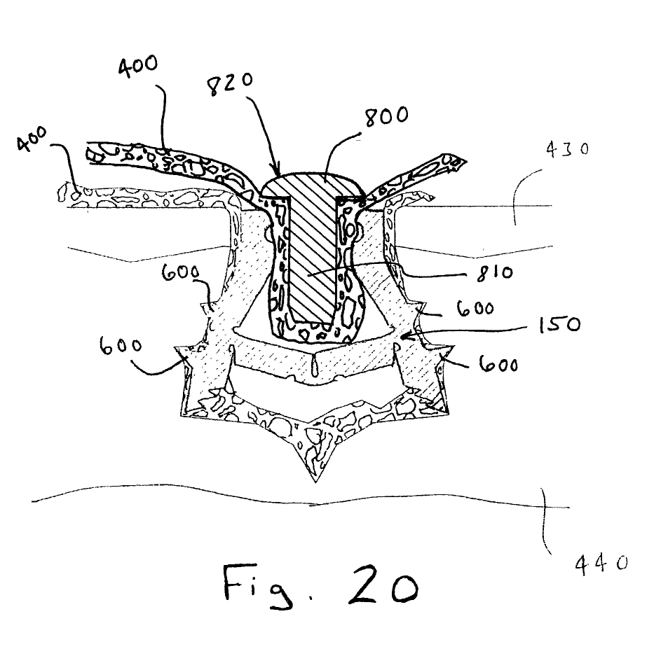

Yet another way to progressively increase tension involves a system similar to that described above with respect to FIG. 19, but does not involve a second anchoring device. A plug having head 800 and shaft 810 could be forced into the first-placed anchoring device as shown in FIG. 20, where plug 820 is shown disposed within anchoring device 150. In this embodiment, plug 820 is simply friction fit into anchoring device 150 and is held in place by being compressed within soft tissue 400. Plug 820 is also made of a biodegradable material. This embodiment requires an additional tool to pull soft tissue over the top opening of anchoring device 150 prior to plug 820 being inserted. This method would also require an additional tool for pushing plug 820 into place. Methods and tools for use in placing such a plug are known to those skilled in the art.

The present invention has been set forth with regard to several preferred embodiments, but the full scope of the invention should be ascertained by the claims that follow.

What is claimed:

1. An anchoring device for attaching soft tissue to bone comprising:
a base; and
two tissue-grabbing members, each tissue-grabbing member having a base end and a tip end with a grasping region disposed between said tip and said base end, the base end connecting each said member to said base.

2. The anchoring device of claim 1 further comprising tissue engaging teeth disposed on the tissue-grabbing members.

3. The anchoring device of claim 1 further comprising bone engaging barbs disposed on the tissue-grabbing members.

4. The anchoring device of claim 1 wherein the device is made from a biodegradable material.

5. The anchoring device of claim 1 wherein the device is made from a material selected from the group consisting of poly(l-lactide), poly(dl-lactide), and combinations thereof.

6. The anchoring device of claim 1 wherein the base has a central hole disposed therein.

7. The anchoring device of claim 1 wherein the base is round.

8. The anchoring device of claim 7 wherein the round base has a central hole disposed therein.

9. An anchoring device for attaching soft tissue to bone comprising:
a base defining a plane;
two tissue-grabbing members, each tissue-grabbing member having a base end and a tip end with a grasping region disposed between said tip and said base end, the base end connecting each said member to said base, said two tissue grabbing members extending in a plane perpendicular to said base; and
means for anchoring the device in bone.

10. The device of claim 9 wherein said anchoring means is an overcenter toggle lock expandable from a collapsed position at which the device can be inserted into bone, to an overcenter stable expanded position to lock the fastener within the bone, said overcenter toggle lock connected to said base and disposed perpendicular to said two tissue-grabbing members.

11. The device of claim 9 wherein:
said base has a hole disposed therein to define a central axis;
said two tissue grabbing members are disposed opposite from each other across the central axis, wherein each said tissue grabbing member has a compression region having a proximate end and a distal end, and an outer dimension spaced from the central axis; and
the outer dimension of the compression region increases in magnitude from the proximal end of the compression region to the distal end of the compression region.

12. The device of claim 10 wherein said overcenter toggle lock has bone engaging barbs.

13. The device of claim 10 wherein said overcenter toggle lock is disposed perpendicular to said tissue grabbing members.

14. An anchoring device for attaching soft tissue to bone comprising:
a base having a hole disposed therein to define a central axis;
an overcenter toggle lock expandable from a collapsed position at which the device can be inserted into bone, to an overcenter stable expanded position to lock the fastener within the bone; and
two tissue grabbing members disposed opposite from each other across the central axis, each said tissue grabbing member having a compression region, the compression region having a proximate end and a distal end, and an outer dimension spaced from the central axis;
the outer dimension of the compression region increasing in magnitude from the proximal end of the compression region to the distal end of the compression region.

15. A system for attaching soft tissue to bone comprising:
(a) an anchoring device comprising:
a base; and
at least two tissue-grabbing members, each said tissue-grabbing member having a base end and a tip end, the base end connecting each said member to said base; and
(b) a delivery device comprising means for expanding said tissue-grabbing members from a grasping position to an expanded position within bone.

16. The system of claim 15, wherein each tissue-grabbing member further comprises a toothed grasping region disposed between said tip end and said base end.

17. The system of claim 15, wherein:
said delivery device comprises:
an applicator having a distal and proximal end; and
an expansion rod removably disposed within the applicator;
wherein the distal end of the applicator is moveable between a first position for holding the anchoring device and a second position for releasing the anchoring device; and
wherein the expansion rod is moveable between a retracted position which corresponds to the first position of the applicator, and a forward position which corresponds to the second position of the applicator.

18. The system of claim 15, wherein said means for expanding comprises a push rod.

19. The system of claim 15, wherein said means for expanding comprises a rotatable screw.

20. A system for attaching soft tissue to bone comprising:
(a) an anchoring device comprising:
a base;
means for anchoring said device to bone; and
two tissue-grabbing members, each tissue-grabbing member having a base end and a tip end with a grasping region disposed between said tip and said base end, the base end connecting each said member to said base; and
b) a delivery device comprising:
an applicator having a distal and proximal end; and
means for expanding said anchoring device;
wherein the distal end of the applicator is moveable between a first position for holding the anchoring device and a second position for releasing the anchoring device; and
wherein said means for expanding is moveable between a retracted position which corresponds to the first position of the applicator, and a forward position which corresponds to the second position of the applicator.

21. The system of claim 20 wherein said anchoring means is an overcenter toggle lock.

22. The system of claim 20, wherein said means for expanding comprises a push rod.

23. A system for attaching soft tissue to bone comprising:
(a) an anchoring device comprising:
a base having a hole disposed therein to define a central axis;
two tissue-grabbing members, each tissue-grabbing member having a base end and a tip end with a grasping region disposed between said tip and said base end, the base end connecting each said member to said base, said two tissue grabbing members extending in a plane perpendicular to said base; and
an overcenter toggle lock expandable from a collapsed position at which the device can be inserted into bone, to an overcenter stable expanded position to lock the fastener within the bone, said overcenter toggle lock connected to said base and disposed perpendicular to said two tissue-grabbing members; and
(b) a second device comprising:
a head; and
a shaft attached to the head; the second device sized to anchor a portion of soft tissue within the hole in the base of the first device.

24. A system for attaching soft tissue to bone comprising:
(a) an anchoring device comprising:
a base;
an overcenter toggle lock expandable from a collapsed position in which the device can be inserted bone, to an overcenter stable expanded condition to lock the fastener within the bone; and
two tissue engaging members; and
(b) a delivery device comprising:
an applicator having a distal and proximal end; and
a push rod slidably and removably disposed within the applicator;
wherein the distal end of the applicator is moveable between a first position for holding the anchoring device and a second position for releasing the anchoring device; and
wherein the push rod is slidable between a retracted position which corresponds to the first position of the applicator, and a forward position which corresponds to the second position of the applicator.

25. The system of claim 24 wherein the delivery device further comprises an outer sleeve slidably and removably disposed around the applicator.

26. The system of claim 24 wherein each of the two tissue engaging members has a compression region having a proximate end and a distal end, and an outer dimension spaced from the central axis, the outer dimension of the compression region increasing in magnitude from the proximal end of the compression region to the distal end of the compression region.

27. The system of claim 25 wherein the outer sleeve is slideable between a retracted position and a forward position, whereby the outer sleeve applies a compression force to the members at their respective compression regions when moved from its retracted position to its forward position.

28. A method for reattaching soft tissue to bone comprising the steps of:
grasping a portion of soft tissue with the distal end of a device;
inserting the device along with the grasped portion of soft tissue into a hole in a bone; and
anchoring the device within the hole into which it was inserted by expanding the device.

29. The method of claim 28 wherein the device is biodegradable.

30. A method for, reattaching soft tissue to bone comprising the steps of:
grasping a portion of soft tissue with the distal end of a device;
inserting the device along with the grasped portion of soft tissue into a hole in a bone;
adjusting the tension on the soft tissue by rotating the device; and
anchoring the device within the hole into which it was inserted by expanding the device after the desired tension is achieved.

31. A method for reattaching soft tissue to bone comprising the steps of:
grasping a portion of soft tissue with the distal end of a device;
inserting the device along with the grasped portion of soft tissue into a hole in a bone;
anchoring the device within the hole into which it was inserted by expanding the device;
grasping a second portion of soft tissue with the distal end of a second device;
inserting the second device along with the grasped portion of soft tissue into a hole in the first device; and
anchoring the second device within the hole in the first device.
